(19) United States
(12) Patent Application Publication
(10) Pub. No.: US 2014/0185780 A1
(43) Pub. Date: Jul. 3, 2014

(54) METHOD AND A SYSTEM FOR AUTOMATICALLY DETERMINING PARAMETERS FOR CONTRAST SCAN

(71) Applicant: GE MEDICAL SYSTEMS GLOBAL TECHNOLOGY COMPANY, LLC., Waukesha, WI (US)

(72) Inventors: Jing ZHAO, Beijing (CN); Yaling TIAN, SiChuan (CN); Ying LIU, Beijing (CN)

(73) Assignee: GE MEDICAL SYSTEMS GLOBAL TECHNOLOGY COMPANY, LLC., Waukesha, WI (US)

(21) Appl. No.: 14/138,201

(22) Filed: Dec. 23, 2013

(30) Foreign Application Priority Data
Dec. 28, 2012 (CN) 201210582011.6

(51) Int. Cl. A61B 6/00

(52) U.S. Cl. CPC A61B 6/545; A61B 6/481
USPC 378/204

(57) ABSTRACT

A method of automatically determining parameters for a contrast scan, the method comprising obtaining scan-object information, selecting a scan protocol corresponding to a part of at least one part of a scan-object to be scanned from a plurality of scan protocols, each of the plurality of scan protocols corresponds to a part of the at least one part of the scan-object to be scanned and comprises built-in scan parameters, and automatically determining a scan delay and injection parameters with respect to the scan-object and the part of the scan-object to be scanned.

### BACKGROUND OF THE INVENTION

1. Field of Invention

Embodiments of the present invention relate to a method and a system for automatically determining parameters for contrast scan.

2. Description of Related Art

Contrast media, also referred to as contrast agents, such as iodine agents, barium sulfate etc., commonly used in X-ray observation, are chemical products that are injected into tissue or part of a human body in order to enhance the effect of influence observation. Densities of these products are higher or lower than those of surrounding tissues, and the formed contrast may be used to be imaged by a specialized instrument.

A contrast scan, also referred to as an enhanced scan, is a scan in CT scan technologies where intravascular contrast media are applied, in which by intravenously injecting contrast media in order to maintain a certain level of contrast media in blood, so that images of a part and a focus are enhanced and displayed more clearly, it facilitates finding the focus that would otherwise be difficult to be found by using CT scan without contrast media, and is mainly used to identify a lesion as vascular or non-vascular, ascertain the relationship between mediastinal lesion and heart great vessel, find out blood supply of the lesion in order to assist in identifying benign and malignant lesions, and so on, which will increase the amount of information of the lesion so as to qualitatively analyze or even distinctly diagnose the lesion.

To minimize the influence of contrast scan on a scan-object and obtain a good contrast scan image, one must properly set scan parameters and injection parameters. Scan parameters include slice thickness, reconstruction interval, pitch, bed speed, scan delay, etc., the scan delay being particularly important, and the injection parameter comprising the name, inject volume and inject rate of the selected contrast medium.

Common contrast media include ionic contrast medium, such as meglucamine diatrizoate, and non-ionic contrast medium, such as Iopromide and ohexol. Different contrast media will bring more or less side reactions to different scan-objects being contrast scanned and parts being scanned, including headache, dizzy, nausea, vomit, urticaria, etc.; and even cause more or less anaphylaxis, including sternutation, lacrimation, conjunctival congestion, face flare, etc., and in a bad way might give rise to throat oedema, pulmonary oedema, hypotension and shock, twitch, stupor, etc. It follows that contrast media should be properly selected based on the body condition of a scan-object and the particular part of a scan-object.

Inject volume and inject rate also have significant influence on the quality of contrast scan. The relationship between inject volume, inject rate and inject duration is: inject volume/inject rate=inject duration. Once inject volume and inject rate are set, inject duration may be calculated based on the relationship, and scan time may be equal to inject duration. The larger the inject volume and the inject rate, the higher the concentration peak value of intravascular contrast media is, the longer the peak duration is, and the more clear the image obtained by scanning is. However, excess inject volume and inject rate will result in side effects, such as a bad reaction of tissue, leakage of blood vessel or even rupture of blood vessel, therefore inject volume and inject rate would not be set too high as well. In general, for a scan-object with good physical condition and target part condition, a higher inject rate and a larger inject volume may be selected, otherwise a lower inject rate and a lesser inject volume may be selected.

In this case, proper setting of scan delay has become the key to contrast scan imaging. The scan delay refers to the interval time from the beginning of injection of contrast media to the starting of scan. If the scan delay is too shoot, the concentration of contrast media at a target part does not reach a peak, whereas if the scan delay is too long, the concentration of contrast media at the target part goes beyond a peak period, both of which will not obtain a good quality contrast scan image, and even some part may not be displayed clearly at all. Only when scan is delayed such that it is synchronized with the period during which the concentration of contrast media within the part reaches a peak, the best imaging effect may be achieved. Proper setting of the scan delay depends on many factors, which may influence the flow rate of contrast media, including (1) inject location, inject volume and inject rate of contrast media, (2) hemodynamics factors of a scan-object, such as heartbeat speed, cardiac output, the presence of arrhythmia, cardiac valve regurgitation, blood vessel stenoses or dilatation, etc., (3) psychological factors of a scan-object, such as anxiety, dread, etc., (4) height, physical condition of a scan-object, and (5) target part being scanned. It follows that in order to obtain the optimized scan delay, these factors should be taken into account as many as possible when the scan delay is set.

There are following three kinds of common contrast scan schemes:

1. General contrast scan (General) scheme, wherein the scan is taken after parameters relating to all of contrast scans are determined.

2. Test Bolus contrast scan (Test) scheme, which sets inject parameters before a test, and uses a low volume injection and low dose test scan during the test to detect the contrast peak time, and then calculates the best scan delay based on the test peak time. With this scheme, it will take two injections and scans, and scan-object suffers more contrast media and radiation dose.

3. Bolus tracking contrast scan (Track) scheme, which first obtains a monitored plane and sets a trigger threshold in a region of interest (ROI), starts the tracking scan around l Os after contrast media are injected, for example, takes one low dose tracking scan and calculates the CT value within ROI every 2 s, automatically triggers the contrast scan when the CT value reaches the threshold. It follows that the scan delay in this scheme appears in the form of threshold, and by setting an appropriate threshold, the scan will of course be triggered automatically following the appropriate scan delay. Since the auto trigger scan technology directly uses the CT value as the trigger threshold, it is more accurate and objective in comparison with the above two schemes. In this scheme, it will take a number of scans and one injection.

However, the above-mentioned schemes currently require that users should set scan parameters and injection parameters by their experiences, which is very inconvenient and inaccurate. For example, general contrast scan scheme currently requires that users should determine all of parameters by their experiences, the reliability of which is low; in Test Bolus contrast scan scheme, users are currently required to set injection parameters by their experiences and calculate the best scan delay by experience formulas; in Bolus tracking contrast scan scheme, users are currently required to set injection parameters and thresholds by their experiences.

Each of conventional steps of setting parameters for the Bolus tracking contrast scan scheme and shortcomings according to embodiments of the present invention thereof will now be specifically explained with reference to existing user interfaces (UI) in FIGS. 1-4.

These steps will be shown with circles in the main UI 100 of FIG. 1: a user clicks “Select New Protocol” button 101 on the main UI 100, which causes a scan protocol selection UI 200 shown in FIG. 2 to be popped-up, to check the scan-object information, wherein the scan-object information is previously entered or obtained from a source of data, such as a database, and then the user will determine the contrast scan related parameters with respect to the scan-object information by his/her experience, and select the scan protocol on the UI 200; the user clicks “Xtream Injector” button 103 on the main UI 100, which causes the Injection parameter setting UI 300 shown in FIG. 3 to be popped-up, to set the Injection parameters; and the user clicks “SmartPrep Rx” button 102 on the main UI 100, which causes the tracking scan parameter setting UI 400 shown in FIG. 4 to be popped-up, to set the tracking scan parameters. These steps are cumbersome and difficult to operate; the setting of parameters in these steps is done by experience and is not pertinent, thereby resulting in inaccurate setting of parameters.

Therefore, it is necessary to provide a method and a system for determining parameters for contrast scan that is easy to use and accurate to set.

### SUMMARY OF INVENTION

Embodiments of the present invention provide a method and a system for determining parameters for contrast scan that is easy to use and is pertinent, in which a number of scan protocols that are in one-to-one correspondence with various parts and have built-in parameters as well as a set of predict models are provided to assist in automatically setting the scan related parameters, thereby avoiding the steps of users determining the contrast scan related parameters, and providing pertinence and accuracy of the parameters.

An embodiment of the present invention provides a method of automatically determining parameters for contrast scan, comprising steps of obtaining the scan-object information; selecting a scan protocol corresponding to the part of the scan-object to be scanned from a number of scan protocols that are in one-to-one correspondence with the parts and have built-in scan parameters; and automatically determining the scan delay and injection parameters with respect to said scan-object and the part to be scanned.

An embodiment of the present invention provides a system for automatically determining parameters for contrast scan, comprising: a means for obtaining the scan-object information; a means for selecting a scan protocol corresponding to the part of the scan-object to be scanned from a number of scan protocols that are in one-to-one correspondence with the parts and have built-in scan parameters; and a means for automatically determining the scan delay and injection parameters with respect to said scan-object and the part to be scanned.

### BRIEF DESCRIPTION OF THE DRAWINGS

Embodiments of the present invention will be described in detail below with reference to the figures of the accompanying drawings which illustrate, by way of example only and not as a limitation, the comparison the embodiments of the present invention with that of the existing technologies, and in which:

FIG. 1 is a main UI used by conventional contrast scan parameter setting methods;

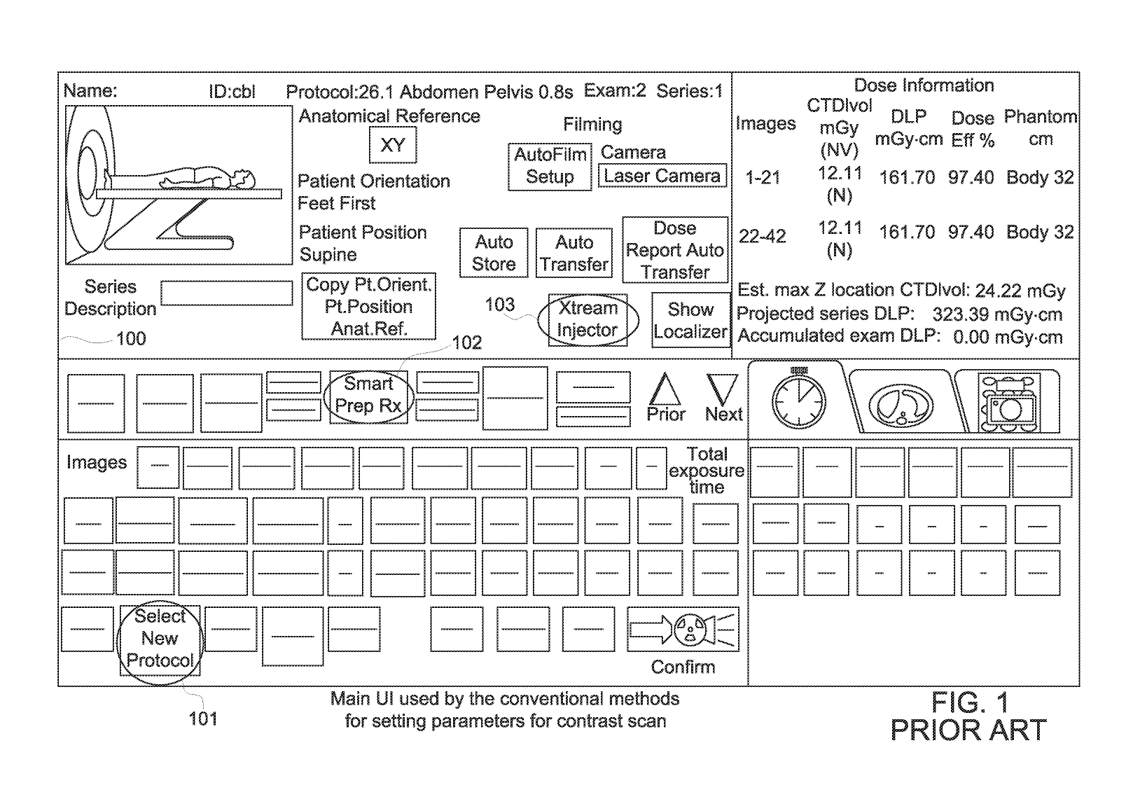

FIG. 2 is a conventional scan protocol selection UI;

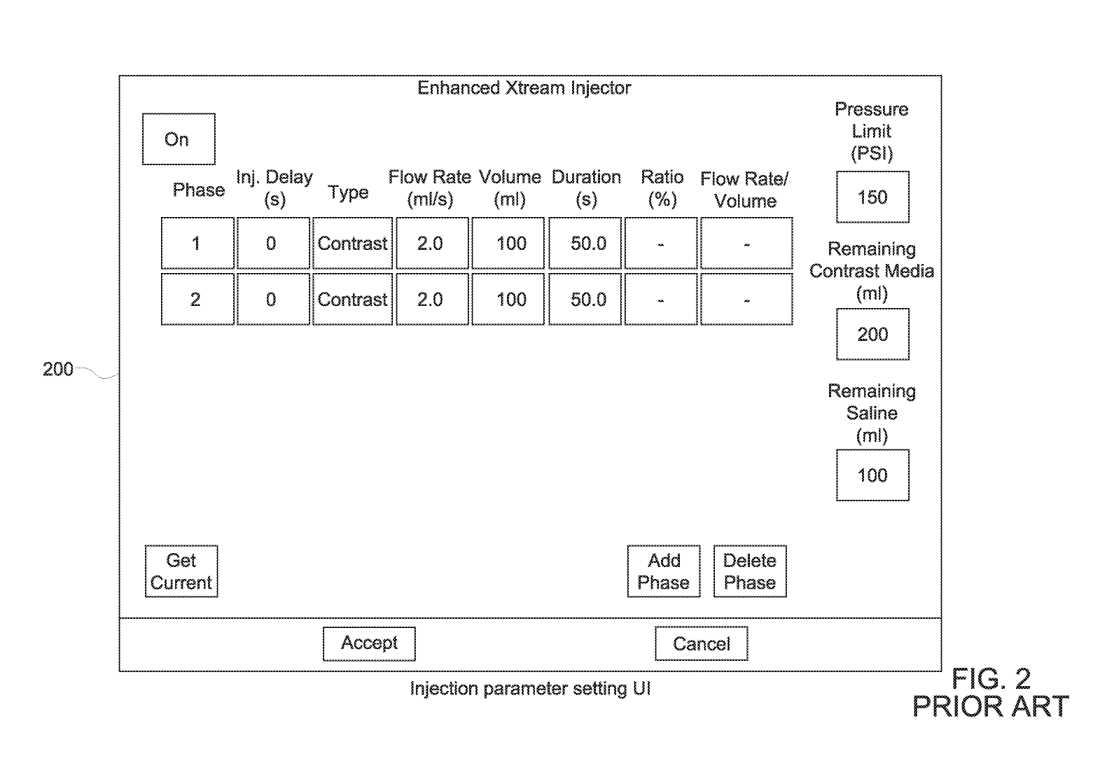

FIG. 3 is a conventional injection parameter setting UI;

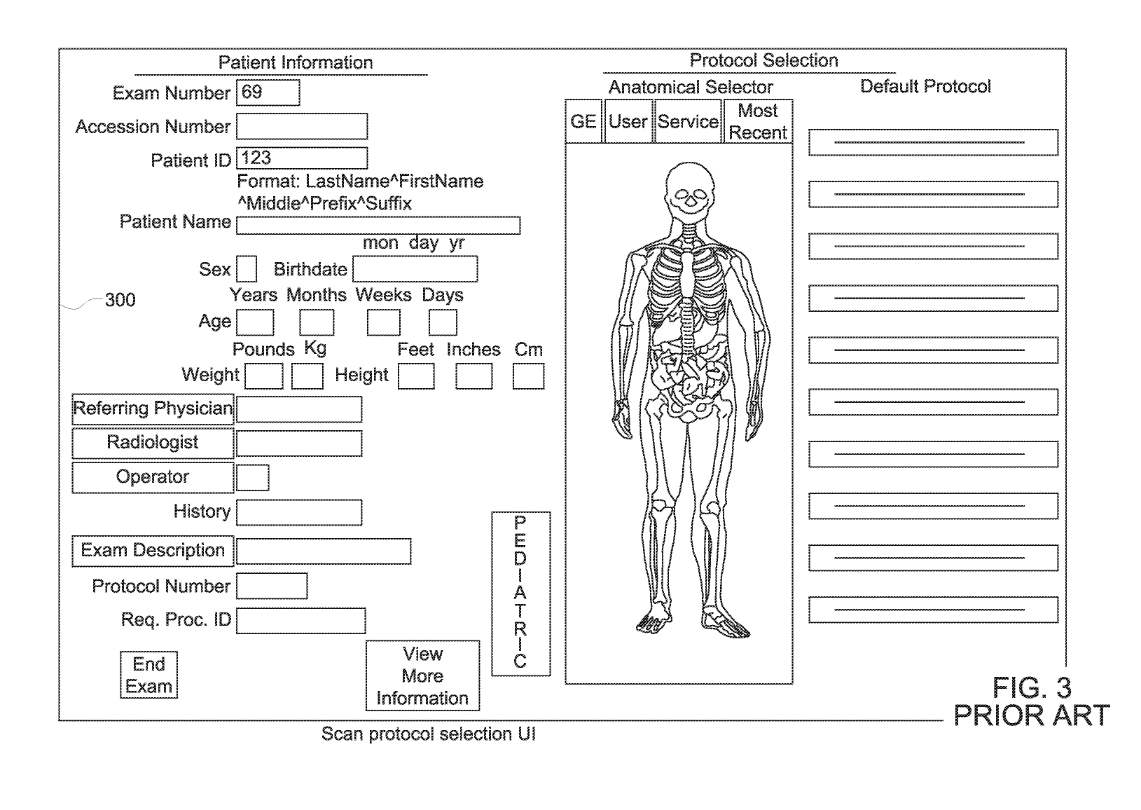

FIG. 4 is a conventional tracking scan parameter setting UI;

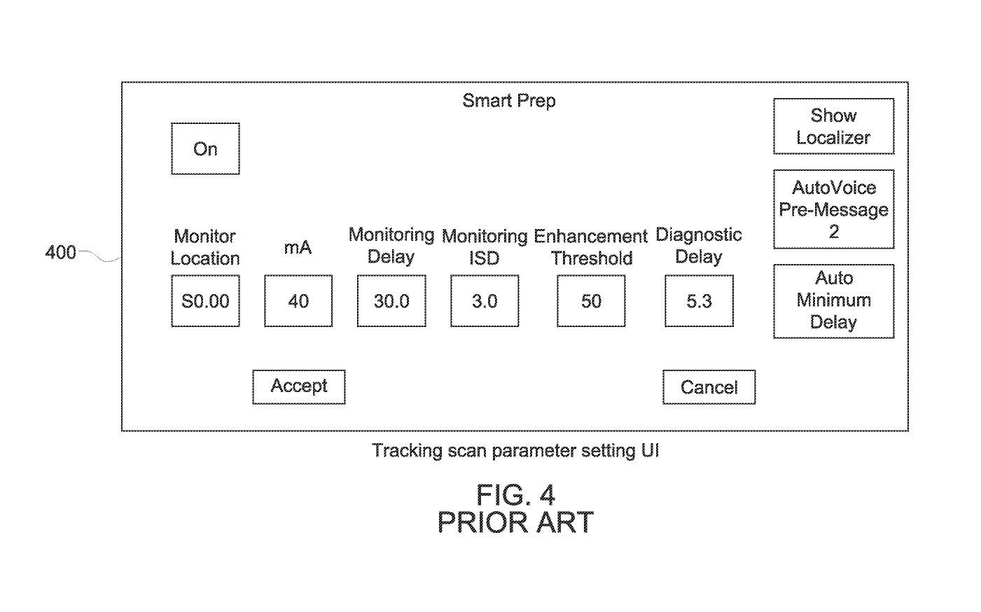

FIG. 5 is a main UI used by the method according to an embodiment of the present invention;

FIG. 6 is a flowchart illustrating the method according to an embodiment of the present invention;

FIG. 7A illustrates a conventional method of setting parameters for general contrast scan;

FIG. 7B illustrates the method according to an embodiment of the present invention;

FIG. 8A illustrates a conventional method of setting parameters for test Bolus contrast scan;

FIG. 8B illustrates the method according to an embodiment of the present invention;

FIG. 9A illustrates a conventional method of setting parameters for Bolus tracking contrast scan; and

FIG. 9B illustrates the method according to an embodiment of the present invention.

### DETAILED DESCRIPTION

As described below, the efficient method of determining parameters for contrast scan set forth according to embodiments of the present invention is pertinent to scan-object and part, in which a number of scan protocols that are in one-to-one correspondence with various parts are provided, and a set of predict models are constructed to assist in predicting the best scan delay and injection parameters, thereby avoiding the steps of users setting these parameters, it is possible even for inexperience users to be able to take contrast scan, thus increasing the efficiency and ease of use, meanwhile, the parameters provided with respect to the specific scan-object and part will also be more accurate.

When an embodiment of the present invention is used to make a decision of contrast scan parameters, after the scan-object information is entered by a user or obtained from a certain source of data, the user will select the scan protocol based on the part of the scan-object to be scanned. In an embodiment of the present invention, since the scan protocols are in one-to-one correspondence with the parts and have built-in scan parameters, the selection of a certain particular scan protocol by a user will be equivalent to the selection of the scan parameters built in the scan protocol, and after the user selects a particular scan protocol, the system may know the corresponding part based on the selected scan protocol without requiring the user enters the part name, thereby, the system will automatically load the scan parameters built in the scan protocol on the UI, and the system also will automatically determine the scan delay and the injection parameters with respect to said scan-object information and said part name. Although the scan parameters built in the scan protocol also include the scan delay, the scan delay determined automatically by the system with respect to the scan-object and part will override the scan delay built in the scan protocol. In an embodiment of the present invention, the scan delay determined automatically by the system will be output in the form of time-Hu curve, the corresponding time period from the beginning of the curve to the maximum peak of the curve is the best scan delay. In an embodiment of the present invention, said injection parameters include the name, inject rate and inject volume of the contrast medium suitable for the scan-object and part. In an embodiment of the present invention, the scan delay and injection parameters will be automatically determined by the predict model with respect to said scan-object information and said part name. In an embodiment of the present invention, the predict model is also in correspondence with the part. In an embodiment of the present invention, a user may also make a further adjustment to the model parameters of the predict model. In an embodiment of the present invention, the model parameters of the predict model include the contrast medium concentration, and in the embodiment, the user may adjust the contrast medium concentration on the advanced setting UI of the predict model. The predict model uses said scan-object information and said part name as input, the best scan delay and injection parameters are calculated and obtained for the specific scan-object and part. In an embodiment of the present invention, the predict model uses pharmacokinetics algorithm or hemodynamics algorithm to perform said calculation, although of course other suitable algorithms may be used by the predict model for said calculation. In an embodiment of the present invention, the user may also make a modification to the algorithm used by the predict model for said calculation. In an embodiment of the present invention, the best scan delay by the predict model calculation is output in the form of time-Hu curve. In an embodiment of the present invention, when the user uses the tracking Bolus contrast scan scheme, the best scan delay output by the predict model may be automatically converted, by the system to the threshold for use in the tracking scan . In an embodiment of the present invention, said automatic conversion is accomplished by the predict model. In an embodiment of the present invention, what is obtain by the predict mode calculation is a set of parameters. In other embodiments of the present invention, multiple sets of parameters are obtained by the predict model calculation, and then a best set of parameters are selected as output from said multiple sets of parameters based on a certain rule. In an embodiment of the present invention, the rule used by the predict model for the above selection is one built in the predict model. In some embodiments of the present invention, the rule used by the predict model for the above selection may be set by the user. The parameters calculated by the predict model will be automatically loaded on the UI, and thus the user will be able to take the scan without manually setting these parameters. In an embodiment of the present invention, the scan parameters and injection parameters loaded automatically on the UI may be further modified by the user.

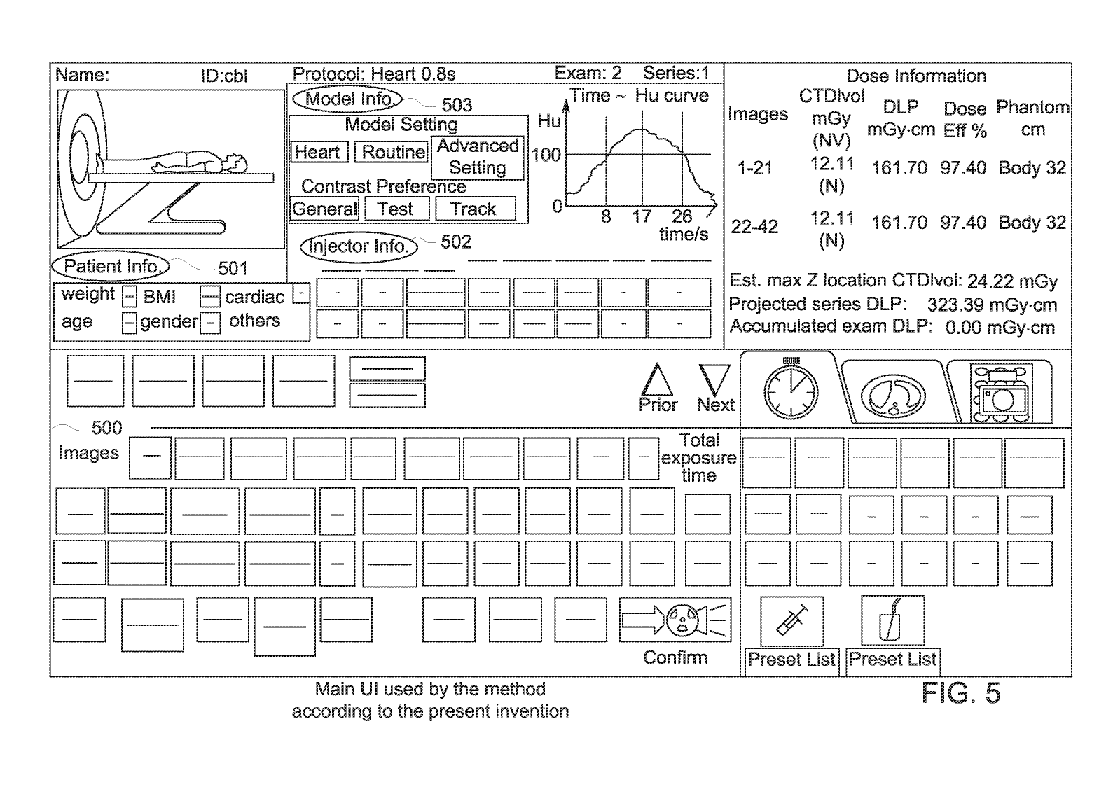

The main UI 500 according to an embodiment of the present invention after the selection of the scan protocol is illustrated in FIG. 5, which is given by way of example only.

Besides the scan parameters, there also are following three kinds of information on the main UI:

(1) Scan-object information 501 (“Patient Info”), this kind of information is pertinent to the part, i.e., a different information is displayed for a different part. The scan-object information is an input to the predict model.

(2) Model information 503 (“Model Info”), including the selected predict model, contrast scan scheme, the Advanced Setting option and the time-Hu curve. “Advanced Setting” button provides an interface for the user to set the model parameters of the predict model and set the tracking information, and the model parameters include the above-mentioned contrast medium concentration, the standard used by the model for selecting the best parameters, the algorithm used by the model calculation, and the like.

(3) Injection information 502 (“Injector Info”), which is a portion of the model output, including the contrast medium name, inject rate and inject volume.

It may be seen from the above description and illustration of the main UI 500, the features of the main UI 500 are as follow: several common contrast scan schemes are integrated in a unified UI frame; modularization information such as scan-object information, injection information and predict model information and the like closely related to the contrast scan are organized in modules; input information is displayed with light color and output information is displayed with dark color, causing users to pay attention to these highlighted key information; and only the contrast scan related data are displayed and irrelevant information are hided until the end of scan to avoid confusing users.

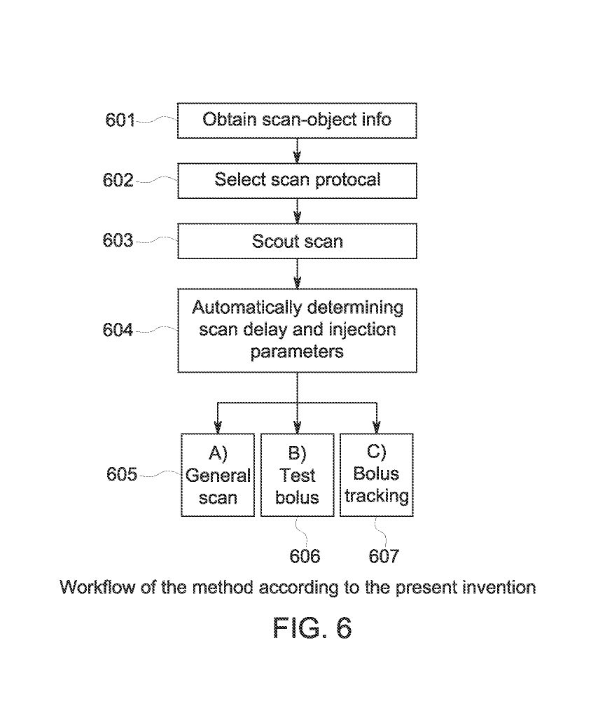

The method according to an embodiment of the present invention will now be described with reference to the main UI 500 and in connection with the flowchart of FIG. 6. In the first step 601, the scan-object information, such as height, weight, age, gender, etc., are entered manually by the user or obtained from a source of data such as database. In the second step 602, which is optional, the user select the scan protocol based on the part of the scan-object to be scanned, as described above, the scan protocols are in one-to-one correspondence with parts and have built-in scan parameters, the selection of the protocol is equivalent to the selection of the part and scan parameters, thus the system determines the part name from the selected protocol, and fills the scan parameters built in the scan protocol on the main UI. In the third step 603, which is optional, the user uses the scan parameters set in the second step to take scout scan, so as to locate the part, if the previously determined part is not in the scan range, the user is prompted to perform position correction for the scan instrument to be aligned with the part. In the fourth step 604, the system automatically determines the scan delay and injection parameters with respect to the scan-object and part, and automatically fills them on the main UI, and in an embodiment of the present invention, some key parameters of the parameters are highlighted to alert the user, and the user may also adjust manually the automatically filled parameters. The fifth step is optional, that is, the user may select to start the scan after the system automatically determines the parameters of contrast scan. In the fifth step, there are three options: a) the user confirms the parameters and take general scan 605 directly; b) the user ignores the scan delay determined automatically in the fourth step 604, but continuously uses some injection parameters and scan parameters determined automatically in the fourth step 604, select “Test” button on the main UI 500 to take test scan 606, in an embodiment of the present invention, following test scan, the system will automatically adjust the scan delay obtained in the test result, and in an embodiment of the present invention, the automatic adjustment is automatically performed by the predict model, and in some embodiments of the present invention, the scan delay obtained in the test result is directly used to take contrast scan; c) Select “Track” button on the main UI 500 to take tracking scan 607, at this point, the system converts the scan delay determined automatically in the fourth step to the threshold for use by tracking scan, and in an embodiment of the present invention, said conversion is accomplished by the predict model, and the contrast scan will be auto triggered after the threshold is reached.

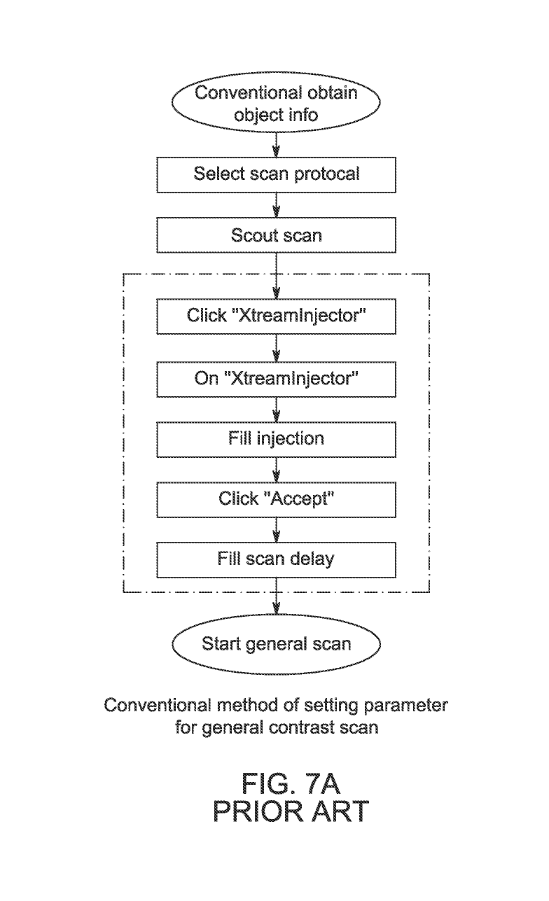

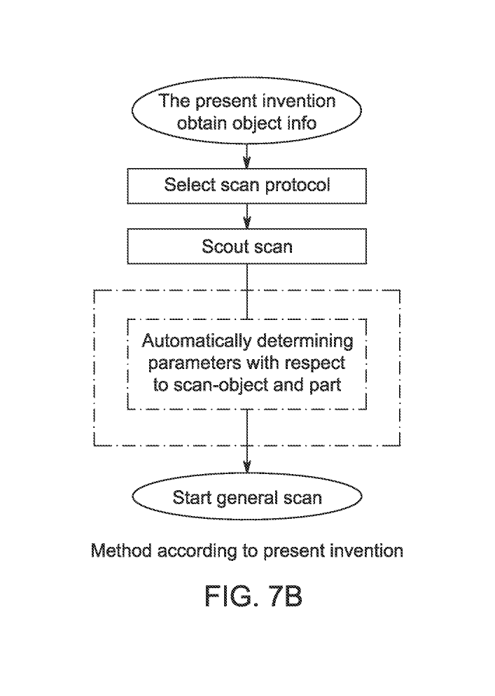

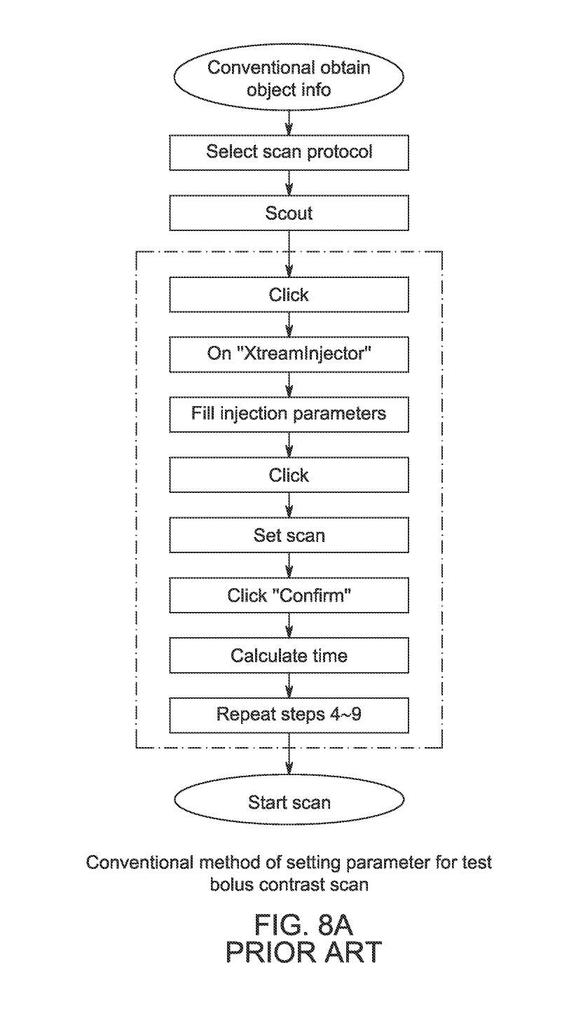

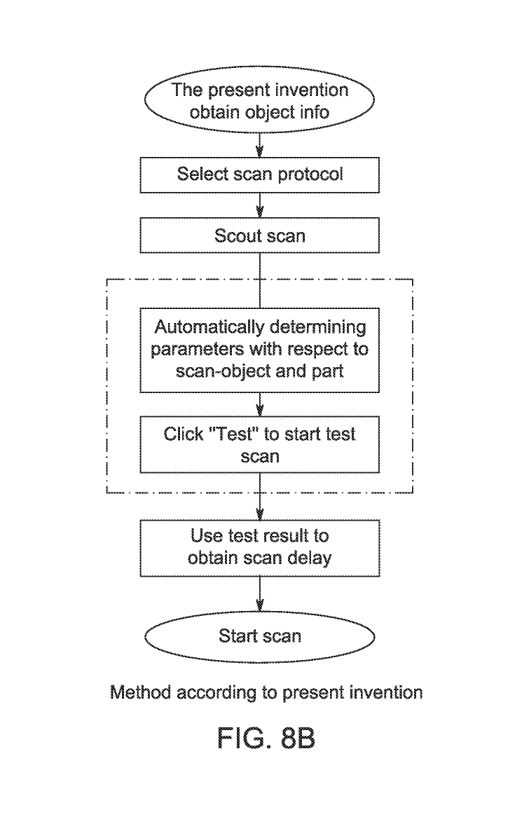

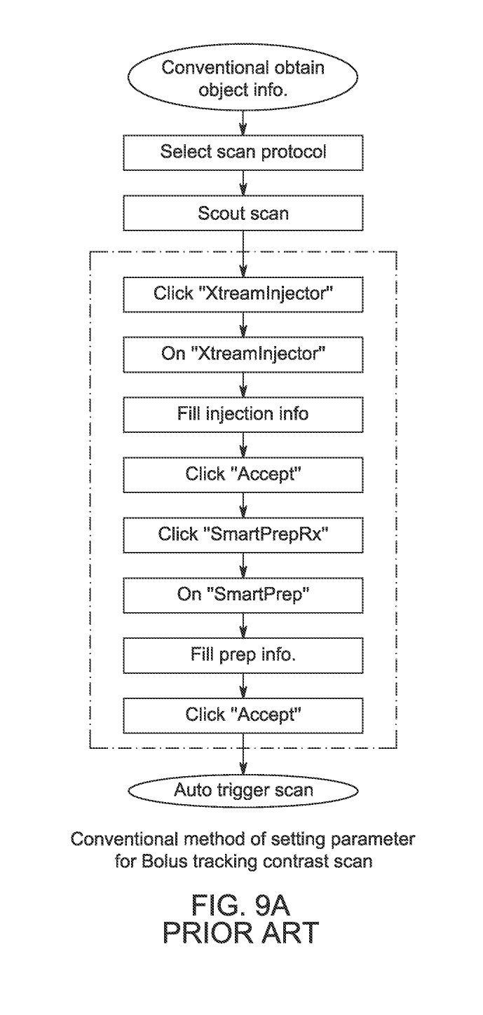

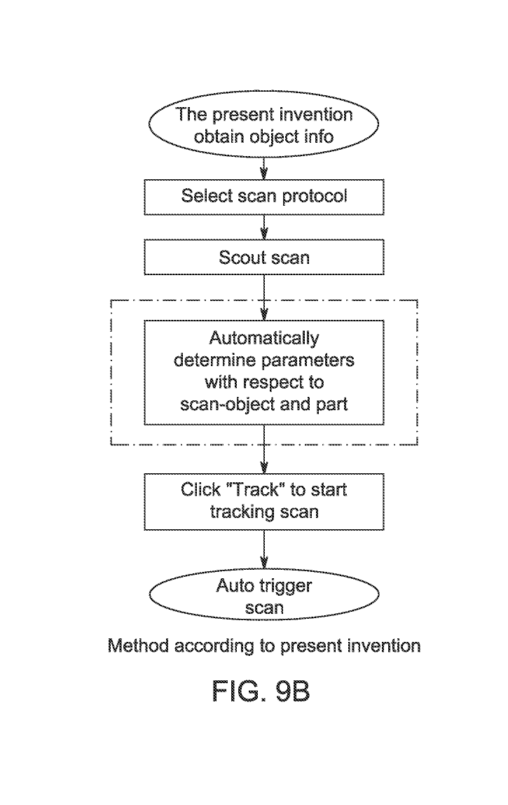

FIGS. 7A, 7B, 8A, 8B, 9A, and 9B illustrate the comparison of conventional methods of setting parameters for various contrast scan schemes with the method according to embodiments of the present invention, with the assumption that an injector has been integrated in the CT system, the auto trigger function has been enabled, and Bolus tracking is achieved by using SmartPrep. In FIG. 7A, the conventional method of setting parameters for general contrast scan requires that the user should click “Xtream Injector” button 103 to invoke the “Xtream Injector” UI 300, fill the injection parameters on the “Xtream Injector” UI , and fill the scan delay on the main UI, whereas the method according to the present invention can automatically set 704 the parameters on the main UI 500. In FIG. 8A, the conventional method of setting parameters for test Bolus contrast scan requires that the user should perform repeat steps of clicking “Xtream Injector” button 103 to invoke the “Xtream Injector” UI 300, filling the injection parameters on the “Xtream Injector” UI 300, and setting the scan parameters on the main UI 100, whereas the method according to the present invention can automatically determine 804 various parameters for test scan and contrast scan. In FIG. 9A, the conventional method of setting parameters for Bolus tracking contrast scan requires that the user should click “Xtream Injector” button 103 to invoke the “Xtream Injector” UI 300, fill the injection parameters on the “Xtream Injector” UI 300, and click “SmartPrepRx” button 102 on the main UI 100 to invoke “SmartPrep” UI 400 and fill Prep information on the “SmartPrep” UI 400, whereas the method according to the present invention can automatically determine 904 various parameters for tracking scan and contrast scan.

It may be seen from the comparison of FIGS. 7A, 7B, 8A, 8B, 9A, and 9B, the number of UIs, the number of steps, number of times of button-click and the number of parameter(s) required to be filled are much lower than those of conventional parameter setting methods, respectively. For one scan phase and one injection phase, the specific numbers are listed in the table below.

| | | | | | Number of | |
|---|---|---|---|---|---|---|
| | | | Number of times | | parameter(s) | |
| | Number of steps | | of button-click | | to be filled | |
| | | The | | The | | The |
| | conven- | present | conven- | present | conven- | present |
| Scheme | tional | invention | tional | invention | tional | invention |
| General | 10 | 5 | 7 | 4 | 3 | 1 |
| Test | 17 | 7 | 11 | 6 | 3 | 1 |
| Track | 13 | 6 | 10 | 5 | 3 | 1 |

Furthermore, since accurate scan delay cannot be obtained in conventional methods of setting parameters for contrast scan, in order to obtain better scan imaging effect, the contrast scan scheme has currently to use the contrast medium with higher concentration (generally, 350˜270 mgI/ml), so that the contrast medium peak will stay longer, while the method according to the present invention may obtain accurate scan delay, so that the contrast scan can maintain the same scan imaging quality while using the contrast medium with lower concentration (about 270 mgI/ml).

Embodiments of the present invention will enable various contrast scan schemes to share the substantially same method workflow and UI frame, avoid some cumbersome steps in conventional methods, it is thus easy for user to use, improves the efficiency, and the scan delay and injection parameters provided automatically by the present invention with respect to the specific scan-object and part will be more accurate than those entered by users according to their experiences and subjective judgment in conventional methods.

Embodiments of the present invention describe a method of automatically determining parameters for contrast scan, comprising steps of: obtaining scan-object information; selecting a scan protocol corresponding to the part of the scan-object to be scanned from a number of scan protocols that are in one-to-one correspondence with parts and have built-in scan parameters; automatically determining the scan delay and injection parameters with respect to the scan-object and the part to be scanned. In an embodiment, the contrast scan refers to general contrast scan. In an embodiment the contrast scan refers to test Bolus contrast scan. In an embodiment the scan delay in the test result is automatically adjusted after the test scan is accomplished. In an embodiment the contrast scan refers to Bolus tracking contrast scan. In an embodiment the scan delay is automatically converted to the threshold for use by tracking scan when tracking scan is started. In an embodiment the scan delay is output in the form of time-Hu curve. In an embodiment the injection parameters include the contrast medium name, inject rate and inject volume. In an embodiment the method further comprises the step of scout scan after the step of selecting the scan protocol and before the step of automatically determining. In an embodiment, the method may further comprise the step of modifying the scan parameters and the injection parameters after the step of automatically determining and before the step of starting scan. In an embodiment the step of automatically determining is performed by a predict model. In an embodiment the predict model is in correspondence with the part. In an embodiment the model parameters of the predict model may be further adjusted. In an embodiment the model parameters include one or more of the contrast medium concentration, the algorithm used by the model and the standard used by the model for determining the best parameters. In an embodiment the predict model uses pharmacokinetics algorithm to perform the step of automatically determining. In an embodiment the predict model uses hemodynamics algorithm to perform the step of automatically determining.

Embodiments of the present invention also describe a system for automatically determining parameters for contrast scan, comprising: means for obtaining scan-object information; means for selecting a scan protocol corresponding to the part of the scan-object to be scanned from a number of scan protocols that are in one-to-one correspondence with parts and have built-in scan parameters; means for automatically determining the scan delay and injection parameters with respect to the scan-object and the part to be scanned. In an embodiment the contrast scan refers to general contrast scan. In an embodiment contrast scan refers to test Bolus contrast scan. In an embodiment the system comprises means for automatically adjusting the scan delay in the test result after the test scan is accomplished. In an embodiment the contrast scan refers to Bolus tracking contrast scan. In an embodiment the system comprises means for automatically converting the scan delay to the threshold for use by tracking scan when tracking scan is started. In an embodiment the scan delay is output in the form of time-Hu curve. In an embodiment the injection parameters include the contrast medium name, inject rate and inject volume. In an embodiment the system further comprises means for taking scout scan after the selection of the scan protocol and before the automatic determination. In an embodiment the system further comprises means for modifying the scan parameters and the injection parameters after the automatic determination and before the starting of scan. In an embodiment the system further comprises the means for automatically determining is a predict model. In an embodiment the predict model is in correspondence with the part. In an embodiment the model parameters of the predict model may be further adjusted. In an embodiment the model parameters include one or more of the contrast medium concentration, the algorithm used by the model and the standard used by the model for determining the best parameters. In an embodiment the predict model uses pharmacokinetics algorithm to perform the automatic determination. In an embodiment the predict model uses hemodynamics algorithm to perform the automatic determination.

Although the present invention has been described with reference to particular embodiments, this description generally aims to set forth the inventive ideas and should not be taken to limit the scope of the present invention, and the scope of the present invention will be defined by the appended claims. Of course, those skilled in the art will also be appreciated that the present invention may be performed in other ways than those specifically described herein, without departing from the basic characteristics of the present invention. The present embodiments are thus to be considered in all respects as illustrative and not restrictive, and all changes which come within the meaning and range of equivalency of the appended claims are intended to included therein.

What is claimed is:

1. A method of automatically determining parameters for a contrast scan, the method comprising:
obtaining scan-object information;
selecting a scan protocol corresponding to a part of at least one part of a scan-object to be scanned from a plurality of scan protocols, each of the plurality of scan protocols corresponds to a part of the at least one part of the scan-object to be scanned and comprises built-in scan parameters; and
automatically determining a scan delay and injection parameters with respect to the scan-object and the part of the scan-object to be scanned.

2. The method according to claim 1, wherein the contrast scan refers to a general contrast scan, a test Bolus contrast scan, or a Bolus tracking contrast scan.

3. The method according to claim 1, wherein the contrast scan refers to a test Bolus contrast scan, wherein the scan delay in a test result is automatically adjusted after a test scan is accomplished.

4. The method according to claim 1, wherein the scan delay is output in the form of time-Hu curve.

5. The method according to claim 1, further comprising scout scanning after selecting the scan protocol and before automatically determining a scan delay.

6. The method according to claim 1, further comprising, after automatically determining a scan delay:
modifying the scan parameters and the injection parameters; and
starting the contrast scan.

7. The method according to claim 1, wherein automatically determining a scan delay comprises performing a predict model.

8. The method according to claim 7, wherein the predict model comprises model parameters, wherein the model parameters comprise one or more of the contrast medium concentration, the algorithm, and the standard used by the predict model for determining the best parameters.

9. The method according to claim 7, wherein performing a predict model comprises using pharmacokinetics algorithm or hemodynamics algorithm.

10. A system for automatically determining parameters for a contrast scan, the system comprising:
a processor configured to:
obtain scan-object information;
receive a selection of a scan protocol corresponding to a part of at least one part of a scan-object to be scanned from a plurality of scan protocols, wherein each of the plurality of scan protocols corresponds to a part of the at least one part of the scan-object to be scanned and comprises built-in scan parameters; and
automatically determine a scan delay and injection parameters with respect to the scan-object and the part of the scan-object to be scanned.

11. The system according to claim 10, wherein the contrast scan refers to a general contrast scan, a test Bolus contrast scan, or a Bolus tracking contrast scan.

12. The system according to claim 10, wherein the contrast scan refers to a Bolus tracking contrast scan, the system further comprising a convertor configured to automatically convert the scan delay to a threshold for use by a tracking scan when the tracking scan is started.

13. The system according to claim 10, wherein the scan delay is output in the form of time-Hu curve.

14. The system according to claim 10, wherein the injection parameters comprise a contrast medium name, an inject rate, and an inject volume.

15. The system according to claim 10, wherein the system is configured to take a scout scan after receiving the selection of the scan protocol and before the automatic determination.

16. The system according to any of claim 10, wherein the processor is further configured to modify the scan parameters and the injection parameters after the automatic determination and before a starting of the contrast scan.

17. The system according to claim 10, wherein the processor is further configured to automatically determine the scan delay based on a predict model.

18. The system according to claim 17, wherein the processor is further configured to adjust model parameters of the predict model.

19. The system according to claim 18, wherein the model parameters comprise one or more of the contrast medium concentration, the algorithm, and the standard used by the predict model for determining the best parameters.

20. The system according to claim 17, wherein the predict model comprises a pharmacokinetics algorithm or a hemodynamics algorithm.
